(19) United States
(12) Patent Application Publication
(10) Pub. No.: US 2023/0106551 A1
(43) Pub. Date: Apr. 6, 2023

(54) GLASS COMPOSITION

(71) Applicant: IR SCIENTIFIC INC., Halifax (CA)

(72) Inventors: Daniel BOYD, Upper Tantallon (CA); Kathleen Naomi MACDONALD-PARSONS, Bedford (CA)

(21) Appl. No.: 17/909,247

(22) PCT Filed: Mar. 2, 2021
(86) PCT No.: PCT/CA2021/050271

§ 371 Date: Sep. 2, 2022

Related U.S. Application Data
(60) Provisional application No. 62/985,207, filed on Mar. 4, 2020.
(60) Provisional application No. 62/984,621, filed on Mar. 3, 2020.

(51) Int. Cl. A61K 8/21; C03C 3/23; C03C 12/00; C03C 4/00; A61K 8/25; A61K 8/24; A61K 8/19; A61Q 11/00

(52) U.S. Cl. CPC A61K 8/21; A61K 8/19; A61K 8/24; A61K 8/25; A61Q 11/00; C03C 3/23; C03C 4/0021; C03C 12/00; A61K 2800/28; C03C 2204/00

(57) ABSTRACT

The present disclosure provides glass compositions that include from 45 mol% to about 95 mol% of B203; from about 3 mol% to about 60 m ol% of one or more glass components selected from the group consisting of: K20, Na20, CaO, and MgO; and from about 2 mol% to about 45 mol% of CaF₂, SnF₂, NaF, KF, Na₂PO₃ F, or a combination thereof, where the glass includes less than 30 mol% of CaF₂, SnF₂, or a combination thereof. The glass includes: substantially no CuO; less than 0.1 mol% of Li₂0, less than 0.1 mol% of Rb₂O, less than 0.1 mol% of BaO; less than 0.1 mol% of P₂0₅; less than 0.1 mol% SiO₂; and less than 30 mol% of MgO. The glass composition may be used to desensitize dentin. The present disclosure also provides dentin-desensitizing compositions, as well as methods and uses of the disclosed glass compositions.

### CROSS REFERENCE TO RELATED APPLICATIONS

This patent application claims the benefit of priority from U.S. Application No. 62/984,621 filed Mar. 3, 2020 and U.S. Application No. 62/985,207 filed Mar. 4, 2020, the contents of which are incorporated herein by reference in their entirety.

### FIELD

The present disclosure relates to glass compositions that may be formulated for dentin-desensitizing compositions.

### BACKGROUND

The following paragraphs are not an admission that anything discussed in them is prior art or part of the knowledge of persons skilled in the art.

Dentin sensitivity is dental pain that arises from exposed dentin surfaces in response to stimuli, such as thermal, evaporative, tactile, osmotic, chemical or electrical. Dentin sensitivity may be caused by gingival recession (receding gums) with exposure of root surfaces, loss of the cementum layer and smear layer, tooth wear, acid erosion, periodontal root planing, or dental bleaching.

Dentine contains many thousands of microscopic tubular structures that radiate outwards from the pulp. Changes in the flow of the plasma-like biological fluid present in the dentinal tubules can trigger mechanoreceptors present on nerves located at the pulpal aspect, thereby eliciting a pain response. This hydrodynamic flow can be increased by cold, air pressure, drying, sugar, sour (dehydrating chemicals), or forces acting onto the tooth. Hot or cold food or drinks, and physical pressure are typical triggers in those individuals with teeth sensitivity.

There is no universally accepted, gold-standard treatment which reliably relieves the pain of dental hypersensitivity in the long term. However, treatments can be divided into in-office (i.e. intended to be applied by a dentist or dental therapist), or treatments which can be carried out at home, available over-the-counter or by prescription.

The purported mechanism of action of these treatments is either occlusion of dentin tubules, or desensitization of nerve fibres/blocking the neural transmission.

### INTRODUCTION

The following introduction is intended to introduce the reader to this specification but not to define any invention. One or more inventions may reside in a combination or sub-combination of the apparatus elements or method steps described below or in other parts of this document. The inventors do not waive or disclaim their rights to any invention or inventions disclosed in this specification merely by not describing such other invention or inventions in the claims.

One example of a dentin-desensitizing composition known in the art is disclosed in PCT Publication No. WO2007144662A1. The disclosed toothpaste includes a bioactive glass comprising strontium. The disclosed bioactive glass occludes dentin tubules and induces precipitation and crystallisation of a carbonated hydroxyapatite. The disclosed bioactive glass is designed to degrade at the same rate as the rate of the induced tissue ingrowth.

One example of a dentin-desensitizing composition known in the art is disclosed in U.S. Pat. No. 5,735,942. The disclosed toothpaste includes a mineral composition composed of CaO, Na₂O, P₂O₅ and SiO₂. The disclosed mineral composition chemically reacts with the surface of dentin and intimately bonds to tooth structure.

One or more described embodiments attempt to address or ameliorate one or more shortcomings involved with dentin-desensitizing compositions that include non-degradable particulate material that occludes dentin tubules. In some embodiments, the disclosed particulate material substantially degrades over a period between 12 and 24 hours under environmental conditions. In some embodiments, the disclosed particulate material provides a controlled release of fluoride over the same time period.

Glass compositions according to the present disclosure include from 45 mol% to about 95 mol% of B₂O₃; from about 3 mol% to about 60 mol% of one or more glass components selected from the group consisting of: K₂O, Na₂O, CaO, and MgO; and from about 2 mol% to about 45 mol% of CaF₂, SnF₂, NaF, KF, Na₂PO₃ F, or a combination thereof. The glass compositions include less than 30 mol% of MgO; less than 30 mol% of CaF₂ or SnF₂; and less than 30 mol% of a combination of CaF₂ and SnF₂.The glass compositions include substantially no CuO; and less than 0.1 mol% of each of Li₂O, Rb₂O, BaO, P₂O₅, and SiO₂.

Glass compositions according to the present disclosure may include B₂O₃ and one or more of MgO and CaO. Such compositions may optionally include one or more of Na₂O and K₂O. In some exemplary compositions, the glass includes B₂O₃, MgO, CaO, and one or more of Na₂O and K₂O.

Glass compositions according to the present disclosure may include one or more of: NaF, KF, and CaF₂, preferably in an amount from about 5 mol% to about 15 mol%.

Glass compositions according to the present disclosure may include: from 45 mol% to about 55 mol% B₂O₃; from about 5 mol% to about 15 mol% K₂O; from about 5 mol% to about 15 mol% Na₂O; from about 10 mol% to about 20 mol% CaO; from about 10 mol% to about 25 mol% MgO; or any combination thereof.

In some examples, glass compositions according to the present disclosure include less than 0.1 mol% of ZnO, and less than 0.1 mol% of SrO. In some embodiments, the glass compositions include substantially no CuO, substantially no Li₂O, substantially no Rb₂O, substantially no BaO, and substantially no P₂O₅.

In some examples, glass compositions according to the present disclosure do not include: (i) from about 5 mol% to about 10 mol% of CaF₂, SnF₂, NaF, KF, Na₂PO₃F, or a combination thereof, and (ii) from about 90 mol% to about 95 mol% of a combination of B₂O₃, Na₂O, MgO, and CaO, where the boron, the magnesium, the combination of sodium and any potassium, and the Ca in the glass composition are present in elemental ratios of about 20: about 4: about 6: about 3, respectively. In some particular examples, glass compositions according to the present disclosure do not include: about 50 mol% B₂O₃; about 15 mol% Na₂O; about 20 mol% MgO; about 10 mol% CaO; and about 5 mol% NaF, KF, CaF₂, SnF₂, or any combination thereof.

In a specific example, a glass composition according to the present disclosure includes 47.6 mol% B₂O₃, 9.5 mol% Na₂O, 14.3 mol% CaO, 19.1 mol% MgO, and 9.5 mol% NaF.

Glass compositions according to the present disclosure may be in the form of a bulk glass, or a particulate material prepared from a bulk glass. The chemical formulations are the same between a bulk glass and the particulate material formed therefrom. The particulate material may include particles that are from about 1 to about 50 µm in size. At least 75% of the particles may be smaller than 50 µm in size, at least 5% of the particles may be smaller than 7 µm in size, or both.

Glass compositions formulated as particulate material may lose at least 5 mass% within 24 hours when exposed to a buffered saline solution. Some exemplary compositions may lose at least 20, at least 40, at least 60, or at least 80 mass% within 24 hours when exposed to a buffered saline solution.

Glass compositions according to the present disclosure may be formulated into a dentin-desensitizing composition, such as a toothpaste, a prophylaxis paste, a tooth varnish, a mouthwash, a dental gel, or a bonding agent. Dentin-desensitizing compositions according to the present disclosure are substantially water-free.

Glass compositions according to the present disclosure may be used for desensitizing dentin, such as in methods that include applying to an individual’s dentin: a toothpaste, a prophylaxis paste, a tooth varnish, a mouthwash, a dental gel, or a bonding agent according to the present disclosure.

### BRIEF DESCRIPTION OF THE DRAWINGS

Embodiments of the present disclosure will now be described, by way of example only, with reference to the attached Figures.

FIG. 1 is an image at 10,000 X from a scanning electron microscope of an exemplary glass composition according to the present disclosure after 30 minutes in simulated body fluid (SBF) at 37° C.

FIG. 2 is an image at 10,000 X from a scanning electron microscope of an exemplary glass composition according to the present disclosure after 3 hours in simulated body fluid (SBF) at 37° C.

FIG. 3 is an image at 10,000 X from a scanning electron microscope of an exemplary glass composition according to the present disclosure after 12 hours in simulated body fluid (SBF) at 37° C.

FIG. 4 is another photograph from a scanning electron microscope of an exemplary glass composition according to the present disclosure after 30 minutes in simulated body fluid (SBF) at 37° C.

FIG. 5 is two images, at 1,000 X and 10,000 X, from a scanning electron microscope of an exemplary glass composition according to the present disclosure after 30 minutes in simulated body fluid (SBF) at 37° C.

FIG. 6 is two images, at 1,000 X and 10,000 X, from a scanning electron microscope of an exemplary glass composition according to the present disclosure after 3 hours in simulated body fluid (SBF) at 37° C.

FIG. 7 is two images, at 1,000 X and 10,000 X, from a scanning electron microscope of an exemplary glass composition according to the present disclosure after 12 hours in simulated body fluid (SBF) at 37° C.

FIG. 8 is two images, at 1,000 X and 10,000 X, from a scanning electron microscope of an exemplary glass composition according to the present disclosure after 24 hours in simulated body fluid (SBF) at 37° C.

FIG. 9 is an image at 10,000 X from a scanning electron microscope of an exemplary glass composition according to the present disclosure after 20 minutes in simulated body fluid (SBF) at 37° C.

FIG. 10 is a set of six images, at 10,000 X collected through energy-dispersive X-ray spectroscopy, showing the mapping of fluoride, sodium, magnesium, phosphorous, calcium and oxygen from an exemplary glass composition according to the present disclosure after 30 minutes in simulated body fluid (SBF) at 37° C.

### DETAILED DESCRIPTION

Glass compositions according to the present disclosure include from 45 mol% to about 95 mol% of B₂O₃; from about 3 mol% to about 60 mol% of one or more glass components selected from the group consisting of: K₂O, Na₂O, CaO, and MgO; and from about 2 mol% to about 45 mol% of CaF₂, SnF₂, NaF, KF, Na₂PO₃F, or a combination thereof, where the glass includes less than 30 mol% of CaF₂, SnF₂, or a combination thereof. The glass includes: substantially no CuO; less than 0.1 mol% of Li₂O, less than 0.1 mol% of Rb₂O, less than 0.1 mol% of BaO; less than 0.1 mol% of P₂O₅; less than 0.1 mol% SiO₂; and less than 30 mol% of MgO.

The glass composition may be formulated as a particulate material that includes particles that are from about 1 to about 50 µm in size. The glass composition may include at least some particles that are sized to luminally occlude dentinal tubules, thereby desensitizing the dentin. In the context of the present disclosure, a particle sized to luminally occlude a dentinal tubule should be understood to mean that the particle sits in or on top of the dentinal tubule, reducing the movement of the dentinal fluid. The glass composition may include at least some particles that are sized to provide surface occlusion of dentinal tubules, thereby desensitizing the dentin.

It should be understood that “about 3 mol% to about 50 mol% of one or more glass components” refers to the total mol% of the glass components, and does not refer to the mol% percent of each individual component. For example, a glass composition according to the present disclosure could include 1 mol% of each of Na₂O, CaO and MgO in order to provide the recited 3 mol% of the additional glass components.

It should be understood that “about X mol%” refers to any value that is within ±2% of the reported percentage. For example, “about 10 mol%” would refer to values from 8 mol% to 12 mol% since all those values would be within ±2% of the reported 10%; and “about 50 mol%” would refer to values from 48 mol% to 52 mol% since all those values would be within ±2% of the reported 50%.

It should be understood that any contemplated range of values is also a disclosure of any value or subrange within the recited range, including endpoints. For example, a contemplated rate of “1 to 100” is also a disclosure of, for example: 1, 10, 25 to 57, 32 to 84, 25 to 84, and 32 to 75.

It should be understood that “about X µm” in the context of particle size is determined based on accepted tolerances as per ASTM E-11 for a test sieve of the noted size. For example, the accepted tolerance for a 50 µm test sieve is 3 µm. Accordingly, “about 50 µm” refers to particles that are from 47 µm to 53 µm in size. In another example, the accepted tolerance for a 35 µm test sieve is 2.6 µm. Accordingly, “about 35 µm” refers to particles that are from 32.4 µm to 38.6 µm in size. The ASTM accepted tolerance for a 25 µm sieve is 2.2 µm. For test sieves without a standard, accepted tolerance (such as test sieves below 20 µm), the expression “about X µm” refers to ±15% for sizes from 5 to 15 µm, and ±50% for sizes less than 5 µm. For example “about 1 µm” refers to particles that are from 0.5 to 1.5 µm in size.

It should be understood that a “glass” according to the present disclosure is a ceramic material that displays a glass transition temperature above room temperature, and whose principal phase is primarily amorphous, such as at least 50% amorphous, at least 75% amorphous, at least 90% amorphous, at least 95% amorphous, or at least 97% amorphous. In some examples, a glass according to the present disclosure is substantially free or completely free, of identifiable crystalline species.

### Glass Compositions

Glass compositions according to the present disclosure include from about 2 mol% to about 45 mol% of CaF₂, SnF₂, NaF, KF, Na₂PO₃F, or a combination thereof, where the glass includes less than 30 mol% of CaF₂, SnF₂, or a combination thereof. Including fluoride in the glass composition results in fluoride being released when the glass degrades. The released fluoride may form fluoridated apatites, such as fluorapatite (Ca₅(PO₄)₃F) in or around the dentinal tubules, which may form a protective precipitate and further decrease dentin sensitivity.

In some examples, the source of fluoride may be from about 5 mol% to about 15 mol%. Compositions that include CaF₂ or SnF₂ provide twice the amount of fluoride per mole of starting material compared to compositions that use NaF, Na₂PO₃F, or KF. In some examples, glass compositions according to the present disclosure may include one or more of: NaF, KF, and CaF₂.

In some examples, glass composition includes sufficient fluoride that 0.1 g of the particulate material releases the fluoride into 10 mL of a buffered saline solution at an average rate of about 0.5 ppm/hr to about 2000 ppm/hr over 1, 2, 4, 8, 12, 18 or 24 hours. In the context of the present disclosure, ppm is measured as mass/volume when determining the release rate of fluoride. In particular examples, the glass composition includes sufficient fluoride that about 4 to about 6 ppm of fluoride is released per hour over 1 hour.

In some examples, glass compositions according to the present disclosure include: B₂O₃, and one or more of MgO and CaO (such as B₂O₃, MgO and CaO). In other examples, glass compositions according to the present disclosure include: (a) B₂O₃, (b) one or more of MgO and CaO, and (c) one or more of Na₂O and K₂O. In still other examples, glass compositions according to the present disclosure include: B₂O₃, MgO, CaO, and one or more of Na₂O and K₂O.

Glass compositions according to the present disclosure may include: from 45 to about 55 mol% B₂O₃; from about 5 to about 15 mol% K₂O; from about 5 to about 15 mol% Na₂O; from about 10 to about 20 mol% CaO; from about 10 to about 25 mol% MgO; or any combination thereof.

Glass compositions according to the present disclosure may include less than 0.1 mol% of ZnO, and less than 0.1 mol% of SrO, such as substantially no ZnO, and substantially no SrO. In particular examples of glass compositions according to the present disclosure, the glass composition includes substantially no CuO, substantially no Li₂O,substantially no Rb₂O, substantially no BaO, substantially no P₂O₅; and substantially no SiO₂. The expression “substantially no” would be understood to mean that the glass composition may include a trace amount of the noted oxide component, but that the glass composition otherwise lacks the noted oxide component.

A glass composition according to the present disclosure may include from about 5 mol% to about 10 mol% of CaF₂, SnF₂, NaF, KF, Na₂PO₃F, or a combination thereof; and from about 90 mol% to about 95 mol% of a combination of B₂O₃, Na₂O, MgO, and CaO; where the boron, the magnesium, the combination of sodium and any potassium, and the Ca in the glass composition are present in elemental ratios of about 20: about 4: about 6: about 3, respectively.

One example of such a glass composition includes: about 50 mol% B₂O₃, about 15 mol% Na₂O, about 20 mol% MgO, about 10 mol% CaO, and about 5 mol% NaF, KF, CaF₂, SnF₂, or any combination thereof.

One specific example of such a glass composition includes: about 50 mol% B₂O₃, about 15 mol% Na₂O, about 20 mol% MgO, about 10 mol% CaO, and about 5 mol% CaF₂. This composition may be referred to herein as composition “PBF1”.

Another specific example of such a glass composition includes: about 48 mol% B₂O₃, about 9 mol% Na₂O, about 19 mol% MgO, about 14 mol% CaO, and about 10 mol% NaF. This composition may be referred to herein as composition “PBF1-Na”.

In a specific example of a PBF1-Na composition, a glass composition according to the present disclosure includes 47.6 mol% B₂O₃, 9.5 mol% Na₂O, 19.1 mol% MgO, 14.3 mol% CaO, and 9.5 mol% NaF.

Some glass compositions according to the present disclosure do not include about 5 mol% to about 10 mol% of CaF₂, SnF₂, NaF, KF, Na₂PO₃F, or a combination thereof; and from about 90 mol% to about 95 mol% of a combination of B₂O₃, Na₂O, MgO, and CaO; where the boron, the magnesium, the combination of sodium and any potassium, and the Ca in the glass composition are present in elemental ratios of about 20: about 4: about 6: about 3, respectively. For example, some glass compositions according to the present disclosure do not include: about 50 mol% B₂O₃; about 15 mol% Na₂O; about 20 mol% MgO; about 10 mol% CaO; and about 5 mol% NaF, KF, CaF₂, SnF₂, or any combination thereof.

### Particle Size Distribution

A glass composition according to the present disclosure may be formulated as a particulate material that includes particles that are from about 1 to about 50 µm in size. Such glass compositions may be referred to as “particulate glass compositions”. In some examples, at least some of the particles are sized to sit in or on top of a dentinal tubule. Dentinal tubules have a natural variation in diameter and are primarily from about 0.5 to about 8 µm in size, for example, from about 0.5 to about 5 µm in size. Accordingly, glass compositions of the present disclosure that are formulated as a particulate material may be used for desensitizing dentin, which may temporarily reduce pain associated with sensitive teeth.

In some examples, at least 75% of the particles making up the particulate material are smaller than 50 µm in size. In other examples, at least 85% or at least 95% of the particles are smaller than 50 µm in size. In some examples, at least 5% of the particles making up the particulate material are smaller than 7 µm in size.

In particular examples, the particulate material is made up of a plurality of particles where at least 5% of the particles are smaller than 35 µm in size, at least 5% of the particles are smaller than 15 µm in size, and at least 5% of the particles are smaller than 7 µm in size.

In particular examples, the particulate material is made up of a plurality of particles where at least 5% of the particles are from about 15 µm to about 35 µm in size, at least 5% of the particles are from about 6 µm to about 15 µm in size, and at least 5% of the particles are from about 3 µm to about 7 µm in size.

In some particular examples, the particulate material is made up of a plurality of particles where the particle size distribution is Dx10 of about 5 um, Dx50 of about 15 um, and Dx90 of about 30 um.

### Degradation

Glass compositions according to the present disclosure degrade under physiological conditions, for example particulate glass compositions according to the present disclosure may lose at least 5 mass% within 24 hours when exposed to a buffered saline solution. In some examples, the glass composition may lose at least 20 mass%, at least 40 mass%, at least 60 mass%, or at least 80 mass% within 24 hours when exposed to the buffered saline solution.

### Surface Microhardness and Remineralization

Glass compositions according to the present disclosure, for example particulate glass compositions according to the present disclosure, may increase surface enamel microhardness. In some examples, a toothpaste, a varnish, or a prophylaxis paste according to the present disclosure may be used to increase surface enamel microhardness. In the context of the present disclosure, an increase in microhardness is in comparison to the surface enamel microhardness before any application of the presently disclosed compositions. In some examples, the surface enamel microhardness may be increased by a greater amount than the increase associated with an otherwise identical toothpaste, varnish, or prophylaxis paste that lacks the glass composition of the present disclosure.

Glass compositions according to the present disclosure, for example particulate glass compositions according to the present disclosure, may remineralize surface enamel. Without wishing to be bound by theory, the authors of the present disclosure believe that this remineralization may at least partially contribute to the increase in surface enamel microhardness.

In some examples, a toothpaste, a varnish, or a prophylaxis paste according to the present disclosure may be used to at least partially remineralize surface enamel. In the context of the present disclosure, any remineralization of the surface enamel is in comparison to the surface enamel mineralization before any application of the presently disclosed compositions. In some examples, the surface enamel may be remineralized by a greater amount than the remineralization associated with an otherwise identical toothpaste, varnish, or prophylaxis paste that lacks the glass composition of the present disclosure.

The toothpaste according to the present disclosure may be applied to the enamel of an individual, such as for a period of 30 seconds to 2 minutes, once or twice daily. In some individuals, the surface enamel microhardness may be increased after about two, three, or four days. In other individuals, the surface enamel microhardness may be increased after five days or more. In some individuals, the surface enamel may be at least partially remineralized after about two, three, or four days. In other individuals, the surface enamel may be at least partially remineralized after five days or more.

### Dentin-Desensitizing Compositions

Particulate glass compositions according to the present disclosure may be formulated in a dentin-desensitizing composition that includes a water-free, orally-compatible carrier. Such dentin-desensitizing compositions according to the present disclosure are free of water since the glass composition degrades if exposed to water.

In the context of the present disclosure, “water-free” or “free of water” should be understood to mean that the dentin-desensitizing composition includes so little water that the glass composition remains capable of reducing dentin sensitivity over the expected lifespan of the product. The expected lifespan of the product refers to the longest expected time between when the dentin-desensitizing composition was produced and when the dentin-desensitizing composition was completely used up or disposed of.

The orally-compatible carrier used in the dentin-desensitizing composition may be a mouthwash, a carrier formulated to mix with additional components to form a mouthwash, or an orally-compatible viscous carrier, such as a toothpaste, a dental gel, a prophylaxis paste, a tooth varnish, a bonding agent, or a carrier that is formulated to mix with additional components to form a toothpaste. The orally-compatible viscous carrier may have a viscosity from about 100 cP at 30° C. to about 150,000 cp at 30° C.

The dentin-desensitizing composition may include a particulate glass composition according to the present disclosure in a sufficient amount that the desensitizing composition includes about 100 ppm to about 5,000 ppm of the fluoride. In the context of the present disclosure, ppm is measured in mass/mass when determining the concentration of fluoride in a desensitizing composition.

Without wishing to be bound by theory, the authors of the present disclosure believe that some glass compositions according to the present disclosure that include potassium, such as in the form of K₂O, KF, or both, may have beneficial dentin-desensitizing properties. The potassium in such glass composition may increase extracellular potassium ion concentration around nerves found in the dentin tubules. A high level of extracellular potassium ions may depolarise nerve fibre membranes and/or reduce their ability to repolarise, which ameliorates patient pain. In dentin-desensitizing compositions that include an occlusive agent and a separate potassium salt, the occlusive agent may inhibit the potassium salt from accessing the nerve, thereby reducing the ability of the separate potassium salt to ameliorate the patient pain. In contrast, some potassium-containing glass compositions according to the present disclosure may degrade while occluding the dentin tubule, and release sufficient potassium ion inside the dentin tubule that the concentration of potassium is high enough to ameliorate patient pain.

One example of a dentin-desensitizing composition according to the present disclosure is a toothpaste that includes a particulate glass composition according to the present disclosure and: an abrasive; a detergent such as sodium lauryl sulfate; a fluoride source; an antibacterial agent; a flavorant; a remineralizer; a sugar alcohol such as glycerol, sorbitol, or xylitol; another dentin desensitizing agent; a hydrophilic polymer such as polyethylene glycol; or any combination thereof. The particulate glass composition may be from about 0.5 to about 15 mass% of the toothpaste.

One particular example of a dentin-desensitizing composition according to the present disclosure is a toothpaste that includes a particulate glass composition according to the present disclosure and: glycerin, silica, a polyethylene glycol (such as PEG 400), titanium dioxide, a carbomer, and a sweetener (such as potassium acesulfame or sodium saccharin).

Another particular example of a dentin-desensitizing composition according to the present disclosure is a toothpaste that includes a particulate glass composition according to the present disclosure and: α-carbomer, DL-limonene, glycerin, mint flavor, a polyethylene glycol (such as PEG-8), silica, titanium dioxide, sodium lauryl sulphate, and a sweetener (such as potassium acesulfame or sodium saccharin).

Another particular example of a dentin-desensitizing composition according to the present disclosure is a toothpaste that includes a particulate glass composition according to the present disclosure and: glycerin, sodium lauryl sulphate, silica (also referred to as silicon dioxide), Carbopol 940 (a crosslinked polyacrylic acid polymer, also referred to as Carbomer 940), and a flavoring agent (such as spearmint oil). The glycerin may be pure glycerol.

In a specific example, the toothpaste may contain about 85 mass% glycerol, about 1.2 mass% sodium lauryl sulphate, about 7.5 mass% silica, about 0.5 mass% carbopol 940, about 1.0 mass% flavoring agent, and about 5.0 mass% of the particulate glass composition according to the present disclosure. The particulate glass composition may be PBF1-Na, sieved to obtain particles ≤ 25 µm.

Another example of a dentin-desensitizing composition according to the present disclosure is a carrier that includes a particulate glass composition according to the present disclosure, where the carrier is formulated to be mixed with additional components to form a toothpaste.

Yet another example of a dentin-desensitizing composition according to the present disclosure is a carrier formulated to mix with additional components to form a mouthwash. Particular examples of the carrier include a particulate glass composition according to the present disclosure and: a water-free alcohol, cetylpyridinium chloride, chlorhexidine, an essential oil, benzoic acid, a poloxamer, sodium benzoate, a flavor, a coloring, or any combination thereof. The additional component(s) that is/are mixed with the carrier to form the mouthwash may include: water, peroxide, cetylpyridinium chloride, chlorhexidine, an essential oil, alcohol, benzoic acid, a poloxamer, sodium benzoate, a flavouring, a colouring, or any combination thereof. The carrier and the additional components may be kept in separate compartments, and mixed together before the mixture is used as a mouthwash. The separate compartments may be in the form of a multi-chambered bottle, such as a bifurcated bottle.

Another example of a dentin-desensitizing composition according to the present disclosure is a prophylaxis paste (also referred to as a “prophy paste”) that includes a particulate glass composition according to the present disclosure. Particular examples of contemplated prophy pastes include a glass composition according to the present disclosure and: pumice, glycerin, diatomite (preferably fine grit), sodium silicate, methyl salicylate, monosodium phosphate, sodium carboxymethylcellulose, a sweetener (such as potassium acesulfame or sodium saccharin), a flavouring, a colouring, or any combination thereof.

### Methods

Glass compositions according to the present disclosure may be synthesized by: mixing appropriate molar amounts of the starting reagents; packing the precursor blend in a platinum rhodium crucible (XRF Scientific, Perth Australia); placing the packed crucible in a furnace (Carbolite, RHF 14/3) at an initial dwelling temperature of 600 to 750° C.; holding the temperature for 60 minutes; ramping the temperature (such as at a rate of 20° C./minute) to a dwelling temperature of 1,200° C.; holding the temperature for 60 minutes; and quenching the glass melt between two stainless steel plates.

It should be understood that the specific ramp rate, times, and temperatures disclosed above could be modified, so long as the glass melts. Ramp rates from 10-20 degrees/min, and holding at the dwell temperature may remove at least some gas bubbles from the glass.

Although the resulting glass composition includes oxides, the starting reagents may include oxides, carbonates, or both. For example, the starting reagent may include boron oxide, sodium carbonate, and calcium fluoride. The calcium carbonate and sodium carbonate decompose in the furnace to release CO₂, generating their corresponding oxides.

The resulting quenched glasses may be ground/milled separately within a planetary micro mill (Pulverisette 6, Fritsch, Germany) and sieved with ASTM E-11 compliant sieves (Cole Palmer, U.S.A) to obtain particles of ≤25 µm. Glasses may be stored under desiccating conditions in sealed storage vials.

Fluoride release is measured by placing 0.1 g of the particulate glass composition in 10 ml of TRIS buffered saline (BioUltra, Sigma Aldrich, Canada) in a 15 ml Falcon tube. The solution is agitated at 120 rpm and kept at a temperature of 37° C. for the desired release period, such as for 1, 3, 6, 12 or 24 h. On completion, the solids are separated through centrifugation (15 minutes and 1500 RCF) and the liquid is decanted into new clean 15 ml Falcon tubes, which were capped and then stored at 4° C. until the amount of fluoride is quantified. The concentration of the released fluoride is quantified using an Accumet®AB250 pH/ion selective electrode meter equipped with electrode fluoride combination (Accumet®). Standard solutions are prepared using a fluoride analytical standard specifically for ion selective electrodes (NaF, 0.1 M F, Sigma Aldrich, Canada) and calibration curves are retrieved before analysis. Liquid extracts derived from the extraction of each composition were prepared for ion analysis as per the electrode manufactures instructions. The ion concentrations are reported as the average of n=3 ± SD.

In the context of the present disclosure, mass loss of a glass composition is measured by placing 0.1 g of the particulate glass composition in 10 ml of TRIS buffered saline (BioUltra, Sigma Aldrich, Canada) in a weighed 15 ml Falcon tube. The solution is agitated at 120 rpm and kept at a temperature of 37° C. for the desired release period, such as for 1, 3, 6, 12 or 24 h. After the specified time points elapsed, the tubes were removed from the incubator and the solutions were immediately centrifuged at 1500 RCF for 15 minutes. The supernatant was decanted into fresh 15 mL Falcon tubes. The pellets were dried in an oven at 70° C. in their respective Falcon tubes to a constant weight to assess the residual mass of the glass allowing of mass loss calculation.

Scanning electron micrograph analysis was performed using a Phenon PRoX scanning electron microscope (Thermofisher Scientific, Waltham, Mass).

Thermal analysis of the glass samples were completed via DSC 404 F3A-0230, a high-temperature differential scanning calorimeter, with a Silicone Carbide furnace, in Pt/Rh crucibles (NETZSCH Instruments North America, Burlington Massachusetts, USA). Approximately 0.025 grams of the samples were weighed packed in Pt/Rh crucibles. Samples were heated at a rate of 10 K/min from 20 to 900° C., with an acquisition rate of 100 pts/min under Nitrogen (Praxair, Danbury Connecticut, USA) protective gas at a flow rate of 50 mL/min. The Onset Temperature (Tₒ), Inflection Temperature (Tᵢ), Final Temperature (T_{f}), and Crystallization Onset Temperature (Tₚ₁) were determined with the use of Netzsch Proteus Thermal Analysis Software (VERSION 6.1.0). The Glass Transition Temperature reported in Table 3 is taken from the Onset Temperature (Tₒ) of the samples.

### EXAMPLES

The glass compositions shown in Table 1 were all synthesized by: weighing determined amounts of the analytical grade reagents (boron oxide, calcium carbonate, sodium carbonate, magnesium oxide and sodium fluoride) (Sigma Aldrich, Canada). The individual formulations were mixed in a dry powder blender for at least 60 mins to ensure homogeneity. Each precursor blend was placed and packed in 100 mL platinum rhodium crucibles (XRF Scientific, Perth Australia). The pack crucible was then placed in a furnace (Carbolite, RHF 14/3) at an initial dwelling temperature of 600-750° C. and held for 60 minutes. The temperature was then ramped (20° C./minute) to a final dwelling temperature of 1,200° C. and held for 60 minutes. On removal, each glass melt was quenched between two stainless steel plates. The resulting quenched glasses were ground/milled separately within a planetary micro mill (Pulverisette 6, Fritsch, Germany) and sieved with ASTM E-11 compliant sieves (Cole Palmer, U.S.A) to obtain particles of ≤25 µm.

TABLE 1

Exemplary glass compositions according to the present disclosure (components listed in mol%)

| Composition No. | B₂O₃ | Na₂O | CaO | MgO | CaF₂ | NaF | KF |
|---|---|---|---|---|---|---|---|
| 2 | 67.9 | 23.5 | 1.8 | 1.3 | | 1.0 | 4.5 |
| 3 | 54.2 | 2.4 | 9.1 | 11.6 | | 21.7 | 1.0 |
| 4 | 45.0 | 17.7 | 1.0 | 5.3 | | 30.0 | 1.0 |
| 5 | 45.0 | 22.9 | 1.0 | 23.1 | | 3.0 | 4.9 |
| 6 | 68.6 | 1.0 | 23.1 | 1.0 | | 3.0 | 3.3 |
| 7 | 72.5 | 6.9 | 1.0 | 1.0 | | 17.6 | 1.0 |
| 8 | 48.2 | 9.4 | 13.6 | 4.0 | | 11.0 | 13.8 |
| 9 | 45.0 | 22.9 | 1.0 | 23.1 | | 3.0 | 4.9 |
| 10 | 45.0 | 1.0 | 5.0 | 4.0 | | 15.0 | 30.0 |
| 11 | 45.0 | 22.0 | 1.0 | 1.0 | | 1.0 | 30.0 |
| 12 | 52.2 | 1.0 | 1.0 | 1.0 | | 30.0 | 14.8 |
| 13 | 68.6 | 1.0 | 23.1 | 1.0 | | 3.0 | 3.3 |
| 14 | 45.9 | 2.0 | 24.0 | 23.5 | | 3.6 | 1.0 |
| 15 | 45.0 | 1.0 | 1.0 | 27.5 | | 18.7 | 6.7 |
| 16 | 45.0 | 23.3 | 23.6 | 1.0 | | 4.3 | 2.7 |
| 17 | 95.0 | 1.0 | 1.0 | 1.0 | | 1.0 | 1.0 |
| 18 | 46.2 | 1.0 | 26.9 | 1.0 | | 20.2 | 4.6 |
| 19 | 45.0 | 25.3 | 1.0 | 1.0 | | 14.1 | 13.6 |
| 21 | 45.0 | 2.7 | 29.3 | 4.0 | | 1.0 | 18.0 |
| 22 | 72.3 | 1.0 | 1.0 | 1.0 | | 4.8 | 19.8 |
| 23 | 54.2 | 33.3 | 3.4 | 1.0 | | 7.2 | 1.0 |
| 24 | 45.0 | 25.3 | 1.0 | 1.0 | | 14.1 | 13.6 |
| 25 | 46.0 | 1.0 | 50.0 | 1.0 | | 1.0 | 1.0 |
| 26 | 48.5 | 1.0 | 2.0 | 28.6 | | 1.0 | 18.9 |
| 27 | 59.1 | 11.3 | 1.0 | 13.3 | | 1.0 | 14.3 |
| 28 | 68.3 | 2.1 | 1.2 | 23.2 | | 4.2 | 1.0 |
| 29 | 78.4 | 6.5 | 5.2 | 8.0 | | 1.0 | 1.0 |
| 30 | 45.9 | 2.0 | 24.0 | 23.5 | | 3.6 | 1.0 |
| 31 | 45.0 | 23.3 | 23.6 | 1.0 | | 4.3 | 2.7 |
| 32 | 59 | 13.0 | 4.5 | 1 | 22.5 | | |
| 33 | 47.5 | 9.5 | 14.2 | 19 | | 9.5 | |
| 34 | 56 | 13 | | 1 | 30 | | |

Some of the particles of the exemplary glasses of Table 1 were evaluated for fluoride release in a buffered saline solution over 1, 12 and 24 hours using the method discuss above; and for mass loss. The ppm (mass/vol.) values of released fluoride, and the percent mass loss after 1, 12 and 24 hours are shown in Table 2.

TABLE 2

Mean fluoride release (ppm) at 1, 12 and 24 hours, and mass loss at 1, 12 and 24 hours.

| Composition No. | Fluoride Release (ppm) at 1 hour | Fluoride Release (ppm) at 12 hours | Fluoride Release (ppm) at 24 hours | Mass Loss (%) at 1 hour | Mass Loss (%) at 12 hours | Mass Loss (%) at 24 hours |
|---|---|---|---|---|---|---|
| 2 | 168.36 | 25.2 | 165.04 | 99.8 | 98.4 | 98.4 |
| 3 | 360.62 | 318.58 | 337.39 | 53.5 | 79.5 | 79.5 |
| 4 | 1918.14 | 946.90 | 1288.72 | 93.7 | 91.9 | 91.9 |
| 5 | 714.60 | 459.07 | 422.57 | 82.8 | 79.1 | 79.1 |
| 7 | 670.35 | 463.50 | 444.69 | 98.6 | 93.2 | 93.2 |
| 8 | 500 | 448.01 | 365.04 | 61.9 | 73.0 | 73.0 |
| 10 | 1342.92 | 177.7 | 1068.58 | 81.6 | 82.3 | 82.3 |
| 11 | 1725.66 | 394.7 | 2355.09 | 97.8 | 92.4 | 92.4 |
| 12 | 1216.81 | 273.85 | 1695.80 | 96.0 | 95.2 | 95.2 |
| 14 | 39.6 | 46.7 | 49.15 | 47.9 | 61.1 | 61.1 |
| 15 | 674.78 | 98.2 | 446.90 | 55.1 | 81.9 | 81.9 |
| 16 | 112 | 111.5 | 94.7 | 72.5 | 73.5 | 62.7 |
| 18 | 71.9 | 81.1 | 79.5 | 56.9 | 60.8 | 64.7 |
| 19 | 1915.93 | 418.9 | 1890.49 | 96.8 | 99.2 | 99.2 |
| 21 | 82.85 | 66 | 71.5 | 65.5 | 59.7 | 64.3 |
| 22 | 119.35 | 113.7 | 493.36 | 99.7 | 95.9 | 95.9 |
| 23 | 149.2 | 144.7 | 727.88 | 98.0 | 99.4 | 99.4 |
| 24 | 441.2 | 414.5 | 1787.61 | 92.6 | 96.0 | 96.0 |
| 25 | 16.9 | 21.3 | 21.2 | 40.5 | 55.0 | 59.3 |
| 26 | 127.5 | 115.2 | 413.72 | 67.8 | 85.9 | 85.9 |
| 27 | 137.9 | 134.5 | 117.9 | 92.1 | 91.9 | 93.0 |
| 28 | 50.5 | 64.5 | 64.7 | 64.4 | 93.8 | 93.8 |
| 29 | 14.4 | 16.6 | 16.7 | 76.3 | 97.2 | 97.2 |
| 30 | 41.6 | 50.7 | 52.1 | 48.3 | 62.8 | 62.3 |
| 31 | 110.4 | 103.4 | 95.15 | 66.9 | 67.8 | 69.6 |
| 33 | 105 | - | 103.7 | 51.5 | - | 62.6 |

The density of the glass powders were measured using an AccuPyc 1340 helium pycnometer (Micromeritics, USA) equipped with a 1 cm³ insert. Prior to use, a traceable volume standard was used to calibrate the pycnometer. For glass analysis, the insert was packed with approximately 1 gram of glass powder. Each measurement is the mean of 10 readings.

The percentages of amorphous phase of the samples were assessed using a D2 Phaser X-ray diffractometer, with a Cu source, and a Lynxeye linear array detector (Bruker AXS Inc, Maddison Wisconsin, USA). Diffraction spectra of finely ground samples were collected between 2 theta angles for 10 and 60 degrees, with a step size of 0.02 degrees and a dwell time of 2 seconds. The relative volume of amorphous material was calculated by fitting a background curve to the amorphous halo, and calculating the relative intensity of the background corrected reduced area to the uncorrected global area. The percent amorphous phase is related to the percent crystallinity by the equation (% crystallinity) + (% amorphous phase) = 100.

The particles of the exemplary glasses of Table 1 had the following bulk properties:

Table 3

Bulk properties for some exemplary glasses

| Composition No. | Density (g/cm³) | % Crystallinity | Glass Transition Temp (°C) |
|---|---|---|---|
| 2 | 2.2618 | 9 | 472.4 |
| 3 | 2.4033 | 2 | 505.7 |
| 4 | 2.4529 | 3 | 392.4 |
| 5 | 2.4203 | 7.9 | 422.9 |
| 6 | 2.4139 | 28 | 589.2 |
| 7 | 2.1841 | 25.3 | 433.1 |
| 8 | 2.4951 | 17.9 | 429.1 |
| 9 | 2.4451 | 3.5 | 420.4 |
| 10 | 2.4078 | 19 | 381.3 |
| 11 | 2.3601 | 13.1 | 345.3 |
| 12 | 2.3 | 2.3 | 384.2 |
| 13 | - | 19.9 | 589 |
| 14 | 2.5734 | 2 | 566.9 |
| 15 | 2.4514 | 5 | 480.9 |
| 16 | 2.4633 | 4.9 | 430.2 |
| 17 | 1.8766 | 42.9 | 264.4 |
| 18 | 2.495 | 1.1 | 555.1 |
| 19 | 2.396 | 13.6 | 358.9 |
| 21 | 2.507 | 1.9 | 489.8 |
| 22 | 2.1231 | 18.4 | 387.5 |
| 23 | 2.4051 | 3.6 | 419.4 |
| 24 | 2.4205 | 1.4 | 357.7 |
| 25 | 2.7024 | 38.1 | 607.8 |
| 26 | 2.3411 | 4.2 | 509.1 |
| 27 | 2.3228 | 1.7 | 457.6 |
| 28 | 2.2744 | 2.4 | 538.8 |
| 29 | 2.1748 | 2.5 | 476 |
| 30 | 2.5713 | 2.3 | 568.6 |
| 31 | 2.4563 | 7.1 | 582.4 |
| 32 | 2.5781 | 2.5 | 505.0 |
| 33 | 2.5381 | 1.7 | 490.9 |
| 34 | 2.6302 | 5.1 | 496.2 |

The compositions numbered 2 to 19, and 21 to 31 listed in Table 1 reflect a design space. The results of the tested compositions provided the following equations, which may allow for the relative comparison of different compositions and/or which may be useful to identify trends associated with different components of the compositions. While experimental and modeling error prevents absolute prediction of glass properties, the equations may be used to guide and refine glass composition design. When used together, these models may help suggest which factors may be traded off in the tailoring of multi-component compositions within the tested composition space. In the following equations, the values for the listed components are in percentages (not fractions or decimals). For example, 50 mol% of B₂O₃ would be “50” (and not “0.5”).

The crystallinity of a melt may be generally predicted under the tested quench conditions using the following formula:

$$\begin{matrix}\text{Crystalinity} \text{=} \\ \text{0} \text{.395399*} \left[{\text{B}}_{\text{2}} {\text{O}}_{\text{3}}\right] \text{+} \text{6} \text{.41123*} \left[\text{MgO}\right] \text{+} \\ \text{0} \text{.189429*} \left[\text{CaO}\right] \text{-} \text{0} \text{.45475} \left[{\text{*Na}}_{\text{2}} \text{O}\right] \\ \text{+} \text{0} \text{.11216*} \left[\text{KF}\right] \text{-} \text{027423*} \left[\text{NaF}\right] \text{-} \\ \text{0} \text{.10068*} \left[{\text{B}}_{\text{2}} {\text{O}}_{\text{3}}\right] \text{*} \left[\text{MgO}\right] \\ \text{-} \text{0} \text{.09012*} \left[\text{MgO}\right] \text{*} \left[\text{CaO}\right] \text{-} \\ \text{0} \text{.04963*} \left[\text{MgO}\right] \text{*} \left[{\text{Na}}_{\text{2}} \text{O}\right] \text{-} \text{0} \text{.09054*} \left[\text{MgO}\right] \text{*} \left[\text{KF}\right] \\ \text{-} \text{0} \text{.06405*} \left[\text{MgO}\right] \text{*} \left[\text{NaF}\right] \text{.}\end{matrix}$$

The density of a glass may be generally predicted using the following formula:

$$\begin{matrix}\text{ρ = 0} {\text{.018214*[B}}_{2} {\text{O}}_{\text{3}} \text{] + 0} \text{.023091*[MgO] +} \\ \text{0} \text{.034759*[CaO] + 0} {\text{.021377*[Na}}_{\text{2}} \text{O]} \\ \text{+ 0} \text{.026828*[KF] + 0} \text{.029437*[NaF] +} \\ \text{0} {\text{.000148*[B}}_{\text{2}} {\text{O}}_{\text{3}} \text{]*[MgO]+0} {\text{.000184*[B}}_{\text{2}} {\text{O}}_{3} {\text{]*[Na}}_{\text{2}} \text{O]} \\ \text{-0} {\text{.00017 [CaO]*[Na}}_{\text{2}} \text{O]} \text{.}\end{matrix}$$

Glass densities from about 1.3 g/cm³ to about 2.2 g/cm³ may particularly be useful in non-aqueous oral care formulations. Glycerol and silica, which are the primary liquid and solid components of a non-aqueous toothpaste, have densities of 1.3 and 2.2 g/cm³, respectively.

The glass transition temperature (T_{g}) may be generally predicted using the following formula: T_{g}= 5.11431*[B₂O₃] + 6.112305*[MgO]+ 7.69080 *[CaO] 2.40780*[Na₂O] + 1.24655*[KF] + 2.97520*[NaF]. It should be understood that phase separated glasses may present multiple glass transitions, the magnitude of which is not necessarily representative of the volume distribution of the phases. While the above equation predicts the onset of a glass transition, the predicted onset may not be the predominant glass transition of the composition if phase separation occurs. Accordingly, a predicted glass transition temperature may be significantly different from the measured predominant glass transition temperature.

The equation related to percent of mass loss after 1 hour under the tested conditions is:

$${\text{(100 * e}}^{\text{y}} {\text{) / (1 + e}}^{\text{y}} \text{)}$$

$$\begin{matrix}\text{where y = 0} {\text{.086782*[B}}_{\text{2}} {\text{O}}_{\text{3}} \text{] +} \\ \text{0} \text{.179717*[MgO] - 0} \text{.103676*[CaO] +} \\ \text{0} {\text{.014547*[Na}}_{\text{2}} \text{O]-0} \text{.0} \text{.054198*[NaF] -} \\ \text{0} \text{.011137*[KF] - 0} {\text{.005920*[B}}_{\text{2}} {\text{O}}_{\text{3}} \text{]*[MgO]} \text{.}\end{matrix}$$

The equation related to release of fluoride (in ppm) after 1 hour under the tested conditions is:

$${\text{(2000 * e}}^{y} {\text{) / (1 + e}}^{\text{y}} \text{)}$$

$$\begin{matrix}\text{where y = -0} {\text{.05697*[B}}_{\text{2}} {\text{O}}_{\text{3}} \text{] -} \\ \text{0} \text{.01494*[MgO] - 0} \text{.04459[CaO]+} \\ \text{0} {\text{.18275*[Na}}_{\text{2}} \text{O]+ 0} \text{.031136*[KF] + 0} \text{.14157*[NaF] -} \\ \text{0} \text{.00448*[CaO]*[NaF] + 0} {\text{.003851*[Na}}_{\text{2}} \text{O]*[KF]} \text{.}\end{matrix}$$

Although the above equation does not provide an accurate estimate of the amount of fluoride released for all glass compositions, the model may still be useful to predict the relative changes in fluoride release which could be expected to occur with changes of the composition.

A PBF1 composition was synthesized by: weighing 11.60 g of B₂O₃, 5.30 g of Na₂CO₃, 2.69 g of MgO, 3.33 g of CaCO₃, and 0.7 g of CaF₂ (Sigma Aldrich, Canada). The starting materials were mixed for 60 mins to ensure homogeneity. The blend was placed and packed in 50 mL platinum rhodium crucibles (Johnson Matthey, Noble Metals, Pennsylvania). The packed crucible was then placed in a furnace (Carbolite, RHF 1600) at room temperature. The furnace was heated (25° C./minute) to an initial dwelling temperature of 600° C. and held for 60 minutes. The temperature was then ramped (20° C./minute) to a final dwelling temperature of 1,200° C. and held for 60 minutes. On removal, the glass melt was quenched between two stainless steel plates. The resulting quenched glasses were ground/milled separately within a planetary micro mill (Pulverisette 7, Fritsch, Germany) and sieved with ASTM E-11 compliant sieves (Cole Palmer, U.S.A) to obtain particles of ≤25 µm.

Comparative glass compositions (referred to as Comparative Examples (CE) 1 and 2) were synthesized similarly, using: 5.80 g B₂O₃, 23.66 g P₂O₅, 5.30 g Na₂CO₃, 1,34 g MgO, 6.67 g CaCO₃, and 0.70 g CaF₂ to result in: CE1 with about 25 mol% B₂O₃, about 25 mol% P₂O₅, about 15 mol% Na₂O, about 10 mol% MgO, about 20 mol% CaO, and about 5 mol% CaF₂; and 5.80 g B₂O₃, 23.66 g P₂O₅, 7.07 Na₂CO₃, 1.34 g MgO, 5.00 g CaCO₃, and 0.70 g CaF₂ to result in CE2 with about 25 mol% B₂O₃, about 25 mol% P₂O₅, about 20 mol% Na₂O, about 10 mol% MgO, about 15 mol% CaO, and about 5 mol% CaF₂.

The density of the glass powders were measured using an AccuPyc 1340 helium pycnometer (Micromeritics, USA) equipped with a 1 cm³ insert. Prior to use, a traceable volume standard was used to calibrate the pycnometer. For glass analysis, the insert was packed with approximately 1 gram of glass powder. Three samples of each of the glasses were run and each measurement is the mean of 10 readings.

The density of the PBF1 composition was measured as 2.5951 ± 0.0072 g/cm³. The density of CE1 was measured as 2.7079 ± 0.0021 g/cm³. The density of CE2 was measured as 2.6749 ± 0.0013 g/cm³.

The release of fluoride and the loss of mass was measured for PBF1, CE1 and CE2. Samples were prepared in 15 mL conical test tubes (n=3), which were weighed and recorded. 0.1 grams of each glass powder (≤25 microns) were separately weighed out and placed in 10 mL TRIS buffered saline (BioUltra, Sigma Aldrich, Canada) in the weighed 15 mL Falcon tubes. The tubes were sealed with parafilm before being placed in a shaking incubator at 37° C. and agitated at 120 rpm for four separate time points: 5 mins, 30 mins, 1 hr, 3 hrs, 24 hours and 48 hours. After the specified time points elapsed, the tubes were removed from the incubator and the solutions were immediately centrifuged (Eppendorf, Centrifuge 5702) at 1500 RCF for 15 minutes. The pellets were dried in an oven at 50-70° C. in their respective Falcon tubes.

The release of fluoride ions was measured using an Accumet AB250 pH/ion selective meter equipped with an Fluoride electrode (Fisher Scientific). To calibrate the probe, 6 standard solutions were prepared using a fluoride analytical standard specifically for ion selective electrodes (NaF, 0.1 F, Sigma Aldrich, Canada). The sodium fluoride concentrations of the standards were synthesized as follows: 1000 ppm, 100 ppm, 10 ppm, 1 ppm, 0.01 ppm and 0.001 ppm respectively, using TRIS buffered saline (BioUltra, Sigma Aldrich, Canada) as the solvent. TISAB concentrate (4.5 mL) was added to each standard before calibration (as per manufacturer’s instructions). Once the probe was calibrated, the slope of the standard was checked to ensure it was within range from the instructions of use. TISAB concentrate (1.0 mL) was added to the decanted supernatants and were then measured for its fluoride concentrations using the calibrated probe. The ion concentrations are reported as the average ± SD.

The amount of fluoride ion released by the PBF1 composition was measured as: 89±2 ppm at 5 mins; 94±3 ppm at 30 mins; 105±5 ppm at 1 hour; and 94±7 ppm at 3 hours. There was no measurable fluoride ion released by CE1 or CE2.

The loss of mass was calculated by comparing the mass of the dried samples after their exposure to the TRIS buffered saline to the initial mass of the samples. The mass loss for the PBF1 composition was: 42.0±2.1 % after 5 minutes; 47.3±2.7% after 30 minutes; 51.5±4.3% after 1 hour; 41.7±5.7% after 3 hours; 70.1±6.8% after 24 hours; and 100% after 48 hours.

Apatite formation in simulated body fluid was confirmed for the PBF1 composition, but was not evident with CE1 or CE2. Simulated Body Fluid was synthesized as per the methods and instructions published by Kokubo and Takadama (Kokubo, T. and Takadama, H. Biomaterials (2006) 27:15, pp 2907-2915).

1 L batches of SBF were prepared in 1000 mL Nalgene bottle (FEP bottle). The prepared SBF was stored at room temperature for 24 hrs immediately after synthesis to ensure stability before experimental use. The SBF was preserved in a Nalgene bottle with the lid on tightly and kept at 6° C. if not needed immediately (for up to 30 days for experimental use).

As per the TCO4 method (published in Maçon, A.L.B., Kim, T.B., Valliant, E.M. et al. J Mater Sci: Mater Med (2015) 26:115) 0.75 g of glass powder of each glass composition (n=3) was immersed in 50 mL of SBF, as synthesized per above, in polyethylene containers. Containers were then placed in an incubating orbital shaker at 37° C. and agitated at 120 rpm for 3 time points: 30 mins, 3 hrs and 12 hrs. After the time points elapsed, each specimen was vacuum filtered with Whatman 42 or 5 grade filter paper (particle retention of 2.5 µm) to collect the solid material from the solution. The solids were immediately washed with distilled water and acetone to stop any further reaction.

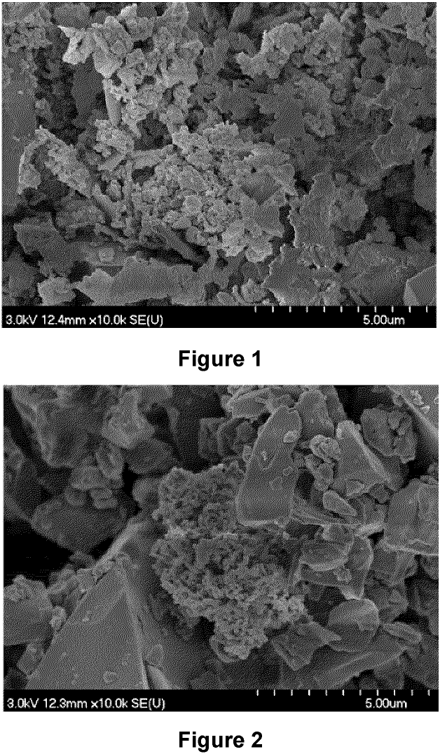

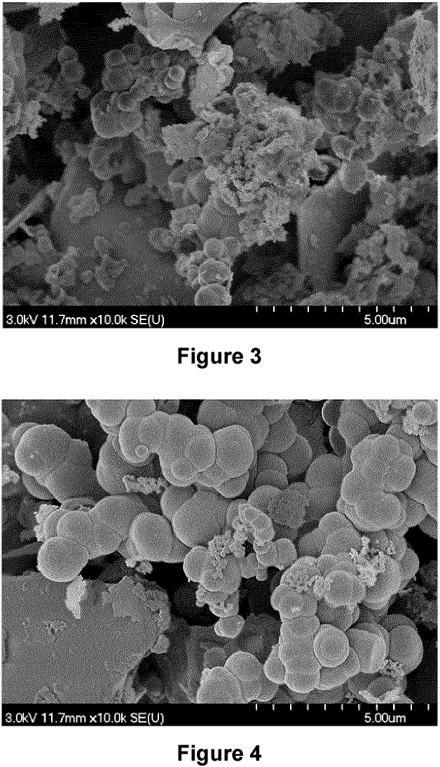

The filtered specimens were dried in a vacuum desiccator for further analysis. Imaging of each specimen was performed using a Hitachi S-4700 FEG (Hitachi, Chula Vista, Ca) scanning electron microscope operating at 3 KV and 15 mA under magnification of 1000 x and 10000 x. Samples were mounted on stubs using double sided carbon tape and sputter coated with gold-palladium for 70 s (Leica EM ACE200, Wetzlar, Germany). The scanning electron microscope images of PBF1 at 30 minutes, 3 hours and 12 hours are shown in FIGS. 1 to 4.

The PBF1 composition was also assessed for dentin tubule occlusion by developing an application protocol and statistical analysis of SEM images graded by two assessors according to a categorical occlusion scale. Sections of human dentin (about 1 to 1.5 mm thick) were prepared from the crowns of caries-free, unrestored molars, perpendicular to the long axis of the roots, using a diamond disc saw. Each section was etched for 2 minutes with 10% citric acid, followed by water rinsing for 60 seconds, sonication for 2 minutes and a further water rinse for 60 seconds. Each section was placed into a 25 mm diameter mould and covered with 3 mm-deep acrylic resin. Once the resin hardened, the dentin face was polished sequentially with 800, and 2500-grit paper to a mirror finish. Following a deionised water rinse, the surface was etched, sonicated and rinsed once more. Sample integrity, tubule density and patency were once again checked under a light microscope, and then with SEM.

A single dentin sample was assigned to each treatment group. The dentin sample was treated with (i) an unformulated mixture of exemplary glass particles, (ii) a test toothpaste that included a mixture of exemplary glass particles, or (iii) a control toothpaste without any additional glass particles. The unformulated mixture was applied using a non-powder nitrile gloved finger for 10 seconds. The test and control toothpastes were applied to the sample with an electric toothbrush for 10 second. The toothpaste was left for 30 seconds before being rinsed until all visible paste was removed. This was repeated for a total of 4 applications of the toothpaste.

Dentin samples were dried in an oven for 1 hour at 37° C., sputter coated with gold, and visualized using a Phenom ProX Scanning Electron Microscope. Five images at x3000 magnification were taken of different portions of each sample, in which the tubules were perpendicular to the surface. Each x3000 micrograph was examined by two single-blinded assessors for the extend of dentin tubule occlusion based on a five-point categorical scale. The grading classification was defined as:
1. Occluded (100% occlusion)
2. Mostly occluded (75% occlusion)
3. Equal (50% occlusion)
4. Mostly unoccluded (25% occlusion)
5. Unoccluded (0% occlusion)

Mean scores for each image were derived from the scores of the two assessors. Standard deviations were calculated, though no formal statistical comparisons were made due to the fact that only one dentin sample was used per treatment group.

Seven different treatment groups were tested, as outlined in Table 4.

Table 4

Treatment groups for test of dentin tubule occlusion

| Treatment Group | Test Article | Materials |
|---|---|---|
| 1 | No treatment of dentin sample | N/A |
| 2 | Test Article #1 | 0.1 g PBF1 |
| 3 | Test Article #2 | 0.0125 g PBF1 + 0.25 g Sensodyne™ Complete Protection (5% w/w) |
| 4 | Test Article #3 | 0.0375 g PBF1 + 0.25 g Sensodyne™ Complete Protection (15% w/w) |
| 5 | Control Article #1 | 0.25 g Sensodyne™ Complete Protection |
| 6 | Control Article #2 | 0.25 g Colgate™ Pro-Relief |
| 7 | Control Article #3 | 0.25 g Sensodyne™ Repair & Protect |

As discussed above, each sample treatment group was tested on a dentin sample and five SEM micrographs of each sample were taken at x3000. Each micrograph was categorically assessed by two assessors. The average score for each micrograph, and the five micrographs per sample, were combined to obtain a group mean score and standard deviation (see Table 5).

Table 5

Mean occlusion score for different treatment groups

| Treatment Group | Group Mean (± SD) |
|---|---|
| 1 | 4.90 (± 0.22) |
| 2 | 1.50 (± 0.0) |
| 3 | 2.90 (± 1.02) |
| 4 | 2.40 (± 0.42) |
| 5 | 3.60 (± 0.22) |
| 6 | 3.60 (± 0.22) |
| 7 | 3.20 (± 0.45) |

The mean baseline score of 4.90 for treatment group 1 illustrates that virtually all dentin tubules were un-occluded. The mean score of 1.50 for the unformulated PBF1 rubbed directly into the dentin sample illustrates a nearly complete tubule blockage. The treatment groups 5, 6 and 7 (control groups lacking PBF1 or any other glass composition according to the present disclosure) had mean occlusion scores from 3.2 to 3.6. The treatment groups 3 and 4 (commercial toothpaste formulated with 5% or 15% PBF1 w/w) had lower mean occlusion scores, indicating a greater degree of tubule occlusion. The degree of occlusion for the commercially available toothpaste Sensodyne™ Complete Protection increased from about 30% occlusion (score 3.6) to about 50% occlusion (score 2.5) when 15% w/w/ of PBF1 was added.

The PBF1 composition was further assessed for dentin tubule occlusion using a 2.5% and 5% w/w PBF1 sodium lauryl sulfate (SLS) paste. In this assessment, the PBF1-toothpastes and control toothpaste were applied to three different dentin samples for each treatment group. The samples were each brushed once for two minutes with the treatment toothpaste. Specifically, each dentin sample was brushed with 0.25 g of a treatment toothpaste for 120 seconds and subsequently rinsed with DI water for 30 seconds. The 2.5% PBF-1 SLS paste resulted in a mean occlusion score of 3.52 ± 0.71. The 5% PBF1-SLS paste resulted in a mean occlusion score of 2.7 ± 0.84. The SLS paste without the PBF1 resulted in a mean occlusion score of 3.80 ± 1.03. A control test using Sensodyne™ Repair & Protect resulted in a mean occlusion score of 3.90 ± 0.66.

A PBF1-Na composition was prepared following the protocols discussed above. Briefly, the glass was synthesized by: weighing 11.05 g of B₂O₃, 3.36 g of Na₂CO₃, 2.56 g of MgO, 4.77 g of CaCO₃, and 1.33 g of NaF (Sigma Aldrich, Canada). The starting materials were mixed for 60 mins to ensure homogeneity. The blend was placed and packed in 50 mL platinum rhodium crucibles (XRF Scientific, Perth Australia). The pack crucible was then placed in a furnace (Carbolite, RHF 14/3) at room temperature. The crucible was placed at initial dwelling temperature of 600° C. and held for 60 minutes. The temperature was then ramped (20° C./minute) to a final dwelling temperature of 1,200° C. and held for 60 minutes. On removal, the glass melt was quenched between two stainless steel plates. The resulting quenched glasses were ground/milled separately within a planetary micro mill (Pulverisette 6, Fritsch, Germany) and sieved with ASTM E-11 compliant sieves (Cole Palmer, U.S.A) to obtain particles of ≤25 µm.

The particle size of ten different samples of the PBF1-Na composition was measured as discussed above.

Table 6

Particle size distribution for PBF1-Na

| | Dx10 (µm) | Dx50 (µm) | Dx90 (µm) |
|---|---|---|---|
| PBF1-Na.1 | 4.7 | 14.9 | 35.2 |
| PBF1-Na.2 | 4.6 | 14.4 | 31.2 |
| PBF1-Na.3 | 4.2 | 13.3 | 29.9 |
| PBF1-Na.4 | 4.5 | 14.1 | 30.7 |
| PBF1-Na.5 | 4.3 | 12.0 | 26.7 |
| PBF1-Na.6 | 4.4 | 13.9 | 30.5 |
| PBF1-Na.7 | 4.1 | 12.1 | 26.2 |
| PBF1-Na.8 | 4.3 | 12.2 | 25.5 |
| PBF1-Na.9 | 4.0 | 11.8 | 25.7 |
| PBF1-Na.10 | 4.2 | 13.3 | 29.7 |
| Average | 4.3 | 13.2 | 29.1 |

The density, % crystallinity, and glass transition temperatures for the ten different samples were also measured as discussed above.

Table 7

Bulk properties for PBF1-Na

| Glass Identifier | Density (g/cm³) | % Crystallinity | Glass Transition Temp (°C) | | |
|---|---|---|---|---|---|
| | | | Onset | Inflection | Fictive |
| PBF1-Na.1 | 2.543(± 0.004) | 1.5 | 494.2 | 508.3 | 518.3 |
| PBF1-Na.2 | 2.537(± 0.004) | 1.8 | 492.6 | 506.1 | 519.6 |
| PBF1-Na.3 | 2.546(± 0.003) | 1.8 | 493.7 | 506.6 | 520.6 |
| PBF1-Na.4 | 2.544(± 0.003) | 1.9 | 493.3 | 506.7 | 519.9 |
| PBF1-Na.5 | 2.548(± 0.004) | 1.8 | 492.5 | 505.6 | 520.0 |
| PBF1-Na.6 | 2.544(± 0.004) | 1.8 | 493.0 | 507.0 | 519.3 |
| PBF1-Na.7 | 2.549(± 0.005) | 1.6 | 491.6 | 504.5 | 517.1 |
| PBF1-Na.8 | 2.546(± 0.004) | 1.8 | 491.3 | 506.6 | 520.4 |
| PBF1-Na.9 | 2.545(± 0.004) | 2.0 | 494.1 | 505.3 | 520.4 |
| PBF1-Na.10 | 2.541(± 0.004) | 1.9 | 491.7 | 506.3 | 517.7 |
| Average | 2.544(± 0.005) | 1.8 | 492.8 | 506.3 | 519.3 |

The mass loss and fluoride release after 24 hours for the ten different samples were also measured as discussed above.

Table 8

Mass loss and fluoride release after 24 hours for PBF1-Na

| Glass Identifier | Mass Loss (%) | Fluoride Release (ppm) |
|---|---|---|
| PBF1-Na.1 | 70.3 | 92.0 |
| PBF1-Na.2 | 71.7 | 91.2 |
| PBF1-Na.3 | 71.7 | 88.9 |
| PBF1-Na.4 | 73.3 | 88.4 |
| PBF1-Na.5 | 73.0 | 87.5 |
| PBF1-Na.6 | 73.3 | 95.0 |
| PBF1-Na.7 | 72.0 | 98.8 |
| PBF1-Na.8 | 72.0 | 93.9 |
| PBF1-Na.9 | 72.7 | 94.2 |
| PBF1-Na.10 | 73.3 | 97.6 |
| Average | 72.3 | 92.8 |

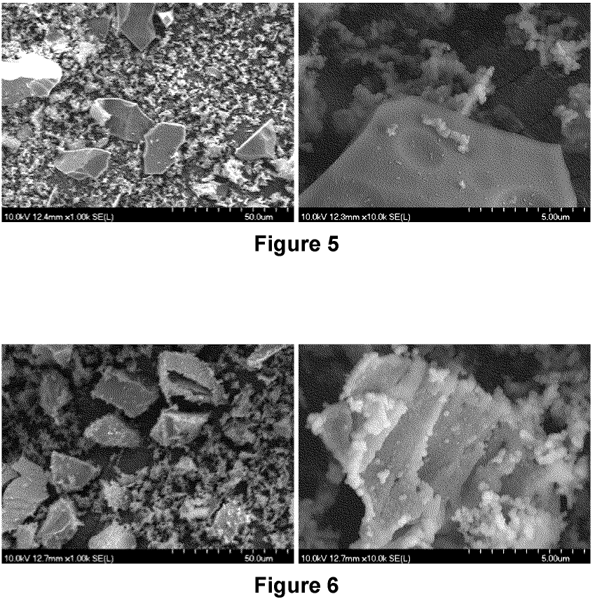

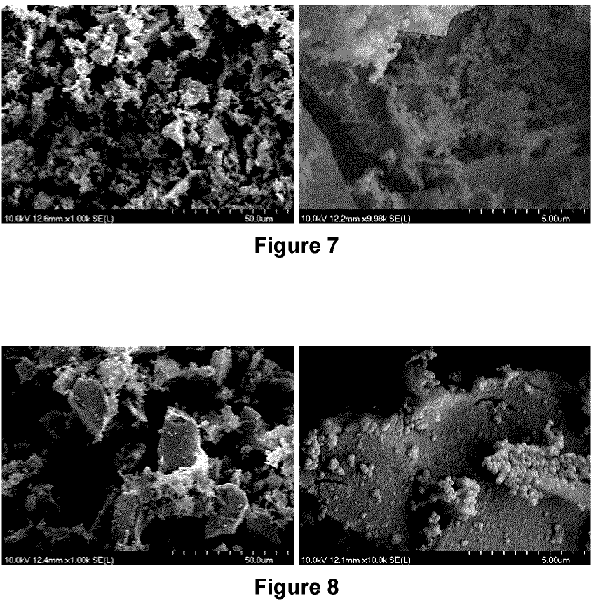

The glass samples were incubated in SBF at 37° C., filtered specimens were dried and imaged using the scanning electron microscope as discussed above. The scanning electron microscope images, at 1,000 X and 10,000 X, of PBF1-Na at 30 minutes, 3 hours, 12 hours and 24 hours are shown in FIGS. 5 to 8.

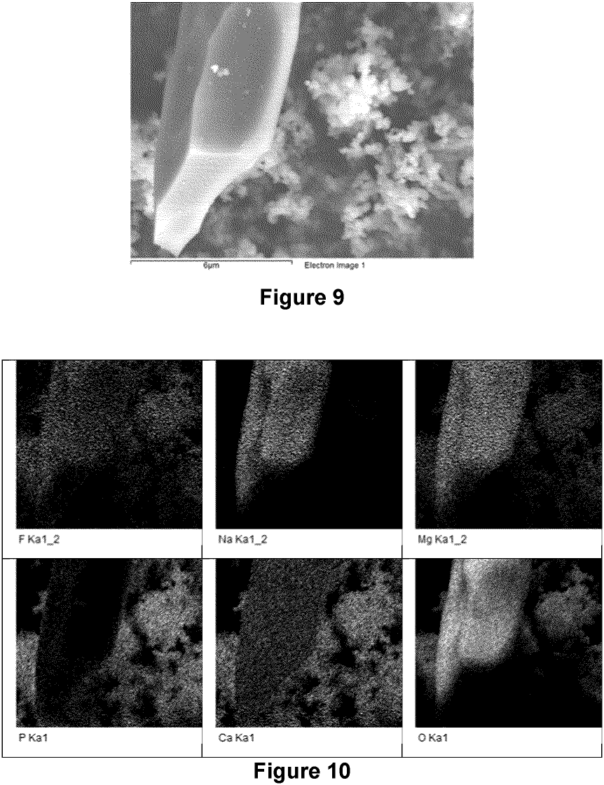

To confirm the identity of precipitates imaged in SEM as fluoridated apatites, Energy-dispersive X-ray spectroscopy was used to map the presence of fluoride, sodium, magnesium, phosphorous, calcium and oxygen (boron mapping was omitted due to the low atomic weight and reduced x-ray interaction associated with light elements). EDS spectra were collected at 10,000 X magnification over 500 counts using the Hitachi S-4700 FEG (Hitachi, Chula Vista, Ca). An SEM of the area of interest for elemental mapping in displayed in FIG. 9, while the element maps are displayed in FIG. 10.

An exemplary toothpaste (“5% SIP-OG”) was prepared using PBF1-Na (specifically, a glass composition consisting of 47.62 mol% B₂O₃ + 9.52 mol% Na₂O + 14.29 mol% CaO + 19.05 mol% MgO + 9.52 mol% NaF) according to the following table:

Table 9

Exemplary toothpaste formulation

| Ingredient | Amount (mass%) |
|---|---|
| Glycerol | 84.80 |
| Sodium Lauryl Sulfate | 1.20 |
| Silicon Dioxide | 7.50 |
| Glass Composition PBF1-Na (particle size ≤ 25 micron) | 5.00 |
| Carbopol 940 | 0.50 |
| Flavour (Spearmint oil) | 1.00 |

In the 5% SIG-OG toothpaste, the glass composition, which consisted of 9.52 mol % of NaF, resulted in a toothpaste with 800 ppm of fluoride.

The glass particles were sieved to collect ≤ 25-micron particles. Particle size analysis confirmed that the powdered particles were appropriately sized to occlude dentin tubules, which typically have diameters from 1 to 5 µm. The mean particle size distribution of the glass was D10 = 4.84 µm, D50 = 14.3 µm, and D90 = 29.80 µm, where Dₓ is the diameter where X% of the distribution has a diameter smaller than the Dₓ.

The exemplary toothpaste 5% SIP-OG was tested in single-time point, and multi-time point dentin occlusion studies.

Single-time point dentin occlusion study. The 5% SIP-OG toothpaste was compared against commercial toothpaste products: (Control Article #1) Sensodyne® Repair and Protect with NOVAMIN® (5% Novamin and 1040 ppm fluoride as sodium fluoride), and (Control Article #2) Colgate® Sensitive PRO-Relief™ (8% Arginine, 35% Calcium carbonate 1320 ppm fluoride as sodium monofluorphosphate) in a single-time point dentin occlusion study.

Analysis of dentin samples treated twice daily using both simulated brushing for 2 minutes, and direct application of a pea-sized amount to an area of sensitivity using a clean finger, provided a measurement of the degree of dentin tubule blockage by the subject toothpastes after one day of treatment. The degree of dentin tubule blockage is commonly understood in the art to be an indirect measure of the ability to reduce dentin hypersensitivity; that is, as the level of occlusion increases, the dentin fluid flow will decrease thereby resulting in decreased sensation of pain. The reduction of dentin fluid flow reduces sensitivity and the precipitation of fluoridated apatites provides a barrier for fast relief. Fluoridated apatites, which help prevent tooth decay or dental caries, may be formed in the presence of fluoride ions in solution, which are incorporated into the mineral.

Human dentin samples (about 1.0 to about 1.5 mm thick) were prepared from the crowns of caries-free unrestored molars, perpendicular to the long axis of the root, using a diamond disc saw. Each section was etched for 2 minutes with 10% citric acid, followed by water rinsing for 60 seconds, sonification for 2 minutes in deionised water, and further rinsed for 60 seconds in water. Each section was placed into a mould and covered with acrylic resin. Once hardened, the dentin face was polished to a mirror finish. Following a rinse with deionised water, the surface was etched, sonicated and rinsed again. Sample integrity, tubule density and patency were verified under scanning electron microscopy (SEM).

Artificial saliva (30 mM potassium chloride, 13 mM sodium chloride, 10 mM potassium dihydrogen orthophosphate, 3 mM calcium chloride dehydrate, 0.22% w/w Type II Porcine Stomach Mucin, and 0.02% w/w sodium azide) was prepared. The dentin samples were immersed in the artificial saliva for at least 60 minutes at 37° C. prior to treatment with the toothpastes.

For brushing application, 0.67 g of toothpaste was applied to the dentin sample using an oscilating Oral-B Precision toothbrush for 10 seconds. For direct application, 0.25 g of toothpaste was applied to the dentin sample using light pressure and a gloved finger for 10 seconds in circular motions. The dentin sample treatment and application conditions are summarized below in Table 10:

Table 10

| | | |
|---|---|---|
| Method of treatment and application | Brushing Application | Direct Application |
| Number of Treatment Days | 1 | 1 |
| # Treatments/Day | 2 | 2 |
| # Replicate Samples | 4 | 4 |
| Treatment Quantity | 0.67 g | 0.25 g |
| Treatment Duration | 10 sec. | 10 sec. |
| Storage between treatments | Artificial saliva | Artificial saliva |

For both application methods, the samples were rinsed for 30 seconds with deionised water following application to remove visible signs of the toothpaste, then stored in artificial saliva for at least one hour before the application cycle was repeated to simulate twice daily use. Following the second application, samples were treated again in simulated saliva for 60 seconds before drying and preparation for SEM imaging.

Treated dentin samples with gold sputter coating were imaged using an Phenon ProX Scanning Electron Microscope, with 3 images collected at x3000 magnification for each sample. Each SEM image was assessed by two double blinded assessors for the extent of denting occlusion based on a five point categorical scale, using the following grading classification:
1. Occluded
2. Mostly occluded
3. Equal
4. Mostly unoccluded
5. Unoccluded

Data analysis was performed using Minitab 18 software. All treatment groups were assessed to provide descriptive statistics of group mean, standard deviation, minimum, maximum, and number of replicates. All data sets were then tested for normalcy. For data sets which passed the assumption of normalcy, 2- sample t-tests were used to make pairwise comparisons between data sets. For pairings where one of more data sets failed to meet the assumption of normalcy, a Mann-Whitney test was used to make pairwise statistical comparisons. All statistical tests were performed at a 0.05 significance level.

Initial performance data supports that the 5% SIP-OG toothpaste is effective and has the ability to partially occlude dentin tubules. The mean occlusion scores are:

TABLE 11

| Toothpaste | Brushing Application Mean Occlusion Score (± SD)¹ | Direct Application Mean Occlusion Score (± SD) ¹ |
|---|---|---|
| 5% SIP-OG | 3.3 ± 0.7 | 4.2 ± 0.4 |
| Colgate® Sensitive PRO-Relief™ | 3.8 ± 0.3 | 3.8 ± 0.5 |
| Sensodyne® Repair and Protect with NOVAMIN® | 3.9 ± 0.2 | 4.3 ± 0.4 |

¹ Categorical occlusion grading where 1 = Occluded, 2 = Mostly occluded, 3 = Equally occluded and unocculded, 4 = Mostly unoccluded, and 5 = Unoccluded

SEM images of dentin tubules treated with the 5% SIP-OG toothpaste show tubule occlusion both by larger undegraded particles retained within the dentin tubule or on the dentin surface, as well as the development of smaller mineral deposits within the dentin tubule.

In addition to intratubular occlusion, formation of a layer on the exposed dentin surface may obstruct the tubules. As the glass composition degrades, the rate of which is influenced by particle size, beneficial ions are released to promote the formation of apatites, including fluoride containing apatites.

Sensodyne® Repair and Protect with NOVAMIN® was the worst performing toothpaste at occluding dentin tubules for both the brushing and direct application. Marketing literature claims that Sensodyne® Repair and Protect with NOVAMIN® “starts working from week 1” supporting that it may exert more of a build-up effect over several days rather than an immediate benefit as demonstrated here by Sensi-IP®. Independent in vitro studies conducted by the Technical Committee 4 of the International Commission on Glass (TCO4) on the original bioactive glass composition 45S5, which is the basis of the Novamin Technology, found that it took 24 hours to begin to see effects of surface reaction in vitro (J Mater Sci: Mater Med 2015).

In contrast, in vitro testing of 5% SIP-OG using the TCO4 methods showed the first evidence of mineralization could be observed at 30 minutes demonstrating a substantially faster mineralization reaction. This increased rate of reactivity of 5% SIP-OG likely contributes to a dual effect of direct tubule occlusion both by the polymer powder particles, as well as the rapid mineralization of the tooth surface through the promotion of the precipitation of fluoridated apatites.

Multi-time point dentin occlusion study. The 5% SIP-OG toothpaste described above was also compared against commercial toothpaste products: (Control Article #1) Sensodyne® Repair and Protect with NOVAMIN® (5% Novamin and 1040 ppm fluoride as sodium fluoride), and (Control Article #2) Colgate® Sensitive PRO-Relief™ (8% Arginine, 35% Calcium carbonate 1320 ppm fluoride as sodium monofluorphosphate) in a multi-time point dentin occlusion study over 5 simulated treatment days.

Analysis of dentin samples treated twice daily using simulated brushing for 2 minutes for one to five days provided a measurement of the degree of dentin tubule blockage by the subject toothpastes over several days. The degree of dentin tubule blockage is commonly understood in the art to be an indirect measure of the ability to reduce dentin hypersensitivity; that is, as the level of occlusion increases, the dentin fluid flow will decrease thereby resulting in decreased sensation of pain.

Human dentin samples were prepared in the same manner as in the single-time point dentin occlusion study, discussed above.

Artificial saliva (30 mM potassium chloride, 13 mM sodium chloride, 10 mM potassium dihydrogen orthophosphate, 3 mM calcium chloride dehydrate, 0.22% w/w Type II Porcine Stomach Mucin, and 0.02% w/w sodium azide) was prepared. The dentin samples were immersed in the artificial saliva for at least 60 minutes at 37° C. prior to the first treatment with the toothpastes.

Samples were treated with the toothpastes (Table 12) twice daily by brushing with 0.67 g of toothpaste with an oscillating toothbrush for 10 seconds.

Table 12

| Treatment | Treatment Description | # Replicates / Treatment Group | Treatment Groups |
|---|---|---|---|
| Test Article | 5% SIP-OG Paste | 4 | 1, 2, 3, 4, and 5-Days |
| Control Article #1 | Sensodyne® Repair and Protect with NOVAMIN® | 4 | |
| Control Article #2 | Colgate® Sensitive PRO-Relief™ | 4 | |

The samples were treated for one to five days as outlined in Table 13. Samples were rinsed for 30 seconds with deionised water following application to remove visible signs of the toothpaste, then stored in artificial saliva for at least one hour before the application cycle was repeated to simulate twice daily use. Following the twice-daily application, samples were soaked in simulated saliva for 3 hours before being transferred into dampened tissue until the next treatment timepoint.

TABLE 13

| | | | | | |
|---|---|---|---|---|---|
| Treatment Group | 1 | 2 | 3 | 4 | 5 |
| Number of Treatment Days | 1 | 2 | 3 | 4 | 5 |
| # Treatments/Day | 2 | 2 | 2 | 2 | 2 |
| Treatment Quantity | 0.67 g | 0.67 g | 0.67 g | 0.67 g | 0.67 g |
| Treatment Duration | 10 sec. | 10 sec. | 10 sec. | 10 sec. | 10 sec. |
| Storage between treatments | Artificial saliva | Artificial saliva | Artificial saliva | Artificial saliva | Artificial saliva |
| Total # Treatments/TimePoint | 2 | 4 | 6 | 8 | 10 |
| Total # Samples/Treatment | 12 | 12 | 12 | 12 | 12 |

Treated dentin samples with gold sputter coating were imaged using an Phenon ProX Scanning Electron Microscope, with 3 images collected at x3000 magnification for each sample. Each SEM image was assessed by two double blinded assessors for the extent of denting occlusion based on a five point categorical scale, using the following grading classification:
1. Occluded
2. Mostly occluded
3. Equal
4. Mostly unoccluded
5. Unoccluded

Data analysis was performed using Minitab 18 software. All treatment groups were assessed to provide descriptive statistics of group mean, standard deviation, minimum, maximum, and number of replicates. All data sets were then tested for normalcy. For data sets which passed the assumption of normalcy, 2- sample t-tests were used to make pairwise comparisons between data sets. For pairings where one of more data sets failed to meet the assumption of normalcy, a Mann-Whitney test was used to make pairwise statistical comparisons. All statistical tests were performed at a 0.05 significance level.

Initial performance data supports that the 5% SIP-OG toothpaste is effective and has the ability to partially occlude dentin tubules. The mean occlusion scores are:

TABLE 14

Mean occlusion scores (+/- SD) for each toothpaste after 1, 2, 3 and 4 days of application (ranging from 1 for fully occluded to 5 for unoccluded)

| Toothpaste | Day 1 | Day 2 | Day 3 | Day 4 | Day 5 |
|---|---|---|---|---|---|
| 5% SIP-OG | 2.6 (+/- 0.6) | 2.0 (+/- 0.4) | 1.2 (+/- 0.4) | 1.4 (+/- 0.5) | 1.0 (+/- 0.1) |
| Colgate® Sensitive PRO-Relief™ | 4.4 (+/- 0.4) | 4.0 (+/- 0.7) | 3.7 (+/- 0.4) | 3.2 (+/- 0.6) | 3.1 (+/- 0.5) |
| Sensodyne® Repair and Protect with NOVAMIN® | 4.0 (+/- 0.6) | 4.0 (+/- 0.1) | 3.2 (+/- 0.8) | 3.5 (+/- 0.5) | 3.9 (+/- 0.8) |

Full occlusion (represented by occlusion scores of 1) was achieved by some Sensi-IP® toothpaste treated dentin samples after 2 days of application of 5% SIP-OG toothpaste. No other toothpastes achieved an occlusion score of 1 for any of the samples treated over the treatment period.

Sensodyne® Repair and Protect with NOVAMIN® and Colgate®Sensitive PRO-Relief™ demonstrated equivalent performance over all timepoints and were inferior to the 5% SIP-OG toothpaste for providing visual occlusion.

Surface Microhardness. Enamel blocks shaped to approximately 4 by 4 mm were sliced from labial bovine incisors, lapped and polished to a grit of 0.04 µm. One corner was abraded off to allow for sample orientation, and samples were stored, refrigerated, and dampened with 0.1% thymol until use.

Baseline surface microhardness measurements were assessed using the Wilson Tukon 1202 microhardness tester. A series of 8 indentations were made at 100 µm spacing, using a 50 g load and 10 second dwell time. Measurement of indent size was performed using an 50 X objective. Samples were accepted into the study with an inclusion criterion of a SMH of ≥ 250 HK, and standard deviation of ≤ 20 HK. Following baseline assessment, an initial demineralization challenge was applied by soaking the samples in 8 ml of demineralization solution per block at 37° C. for 60 minutes, followed by a deionized water rinse. Surface microhardness measurements were taken for each enamel block both before demineralization as a quality check for inclusion in the study, after initial demineralization treatment, and following pH cycling treatment:

TABLE 15

| Step | Solution | Volume | Duration |
|---|---|---|---|
| 1 | Toothpaste slurry | 5 ml | 2 minutes |
| 2 | Remineralizing solution | 20 ml | 58 minutes |
| 3 | Toothpaste slurry | 5 ml | 2 minutes |
| 4 | Remineralizing solution | 20 ml | 58 minutes |
| 5 | Demineralizing solution | 20 ml | 60 minutes |
| 6 | Remineralizing solution | 20 ml | 120 minutes |
| 7 | Toothpaste Slurry | 5 ml | 2 minutes |
| 8 | Remineralizing solution | 20 ml | 58 minutes |
| 9 | Toothpaste Slurry | 5 ml | 2 minutes |
| 10 | Remineralizing solution | 20 ml | Overnight |
| 11 | Repeat Steps 1-10 four more times (5 days total) | | |

A negative control paste was used for comparison, consisting of the equivalent toothpaste chassis without the addition of SIP-OG, along with a positive control which consisted of the equivalent chassis, without SIP-OG, and the addition of 1040 ppm F as NaF.

Surface microhardness (SMH) was analyzed using a series of 8 indents made at 100 µm spacing using a 50 g load and 10 second dwell time. Measurements of the indents was taken using a 50 X objective, and hardness was expressed as Hardness Knoop.

Surface microhardness recovery (SMHR) was calculated using the following equation:

$$\frac{\text{c}}{0} S M H R = 100 x \left[\frac{S M H f i n a l - S M H D e m i n e r a l i z e d}{S M H B a s e l i n e - S M H D e m i n e r a l i z e d .}\right]$$

All statistical analysis was performed using Minitab 18 software. For each experiment, summary statistics were generated for each treatment group and timepoint (n, mean, standard deviation). All data sets were tested for normality using the Anderson-Darling test. Pairwise comparison was performed between treatment groups for each experiment and timepoint. For the enamel surface microhardness experiments, all data sets satisfied the assumptions criteria, and one-way ANOVA was used to compare experimental results. For the visual occlusion experiment, and fluoride uptake tests, 2-sample T tests were used to make pairwise comparisons between occlusion scores when assumptions of normality could be met, and a Mann-Whitney test was used to make comparisons when one or more of the pair failed the normality test. All statistical tests were performed at a 0.05 significance level.

TABLE 16

Mean percentage surface microhardness recovery (+/-) SD for each toothpaste after 5 days of pH cycling treatment.

| Toothpaste | Fluoride content | Mean percentage of SMHR | Statistical comparison groupings |
|---|---|---|---|
| 5% SIP-OG | 800 ppm | 58.8 ± 16.1% | A |
| Positive Control (NaF control) | 1040 ppm | 23.2 ± 24.9% | B |
| Negative Control (blank control) | 0 ppm | - 6.3 ± 14.2% | C |

In the preceding description, for purposes of explanation, numerous details are set forth in order to provide a thorough understanding of the examples. However, it will be apparent to one skilled in the art that these specific details are not required. Accordingly, what has been described is merely illustrative of the application of the described examples and numerous modifications and variations are possible in light of the above teachings.

Since the above description provides examples, it will be appreciated that modifications and variations can be effected to the particular examples by those of skill in the art. Accordingly, the scope of the claims should not be limited by the particular examples set forth herein, but should be construed in a manner consistent with the specification as a whole.

What is claimed is:

1. A glass composition comprising:
from 45 mol% to about 95 mol% of B₂O₃;
from about 3 mol% to about 60 mol% of one or more glass components selected from the group consisting of: K₂O, Na₂O, CaO, and MgO; and
from about 2 mol% to about 45 mol%, such as from about 5 mol% to about 15 mol%, of CaF₂, SnF₂, NaF, KF, Na₂PO₃F, or a combination thereof;
wherein the glass composition comprises:
substantially no CuO;
less than 0.1 mol% of Li₂O;
less than 0.1 mol% of Rb₂O;
less than 0.1 mol% of BaO;
less than 0.1 mol% of P₂O₅;
less than 0.1 mol% SiO₂;
less than 30 mol% of MgO;
less than 30 mol% of CaF₂ or SnF₂; and
less than 30 mol% of a combination of CaF₂ and SnF₂.

2. The glass composition according to claim 1, wherein the glass composition comprises B₂O₃, and one or more of MgO and CaO.

3. The glass composition according to claim 1, wherein the glass composition comprises (a) B₂O₃, (b) one or more of MgO and CaO, and (c) one or more of Na₂O and K₂O.

4. The glass composition according to claim 1, wherein the glass composition comprises B₂O₃, MgO, CaO, and one or more of Na₂O and K₂O.

5. The glass composition according to any one of claims 1 to 4, wherein the glass composition comprises one or more of: NaF, KF, and CaF₂, preferably in an amount from about 5 mol% to about 15 mol%.

6. The glass composition according to claim 1 or 5, wherein the glass composition comprises from 45 mol% to about 55 mol% B₂O₃.

7. The glass composition according to any one of claims 1 to 6, wherein the glass composition comprises from about 5 mol% to about 15 mol% K₂O.

8. The glass composition according to any one of claims 1 to 7, wherein the glass composition comprises from about 5 mol% to about 15 mol% Na₂O.

9. The glass composition according to any one of claims 1 to 8, wherein the glass composition comprises from about 10 mol% to about 20 mol% CaO.

10. The glass composition according to any one of claims 1 to 9, wherein the glass composition comprises from about 10 mol% to about 25 mol% MgO.

11. The glass composition according to any one of claims 1 to 10, wherein the glass composition comprises less than 0.1 mol% of ZnO, and less than 0.1 mol% of SrO, such as substantially no ZnO, and substantially no SrO.

12. The glass composition according to any one of claims 1 to 11, wherein the glass composition comprises substantially no CuO, substantially no Li₂O, substantially no Rb₂O, substantially no BaO, and substantially no P₂O₅.

13. The glass composition according to any one of claims 1 to 12, wherein the composition does not include: from about 5 mol% to about 10 mol% of CaF₂, SnF₂, NaF, KF, Na₂PO₃F, or a combination thereof, and from about 90 mol% to about 95 mol% of a combination of B₂O₃, Na₂O, MgO, and CaO, where the boron, the magnesium, the combination of sodium and any potassium, and the Ca in the glass composition are present in elemental ratios of about 20: about 4: about 6: about 3, respectively.

14. The glass composition according to claim 13, wherein the composition does not include about 50 mol% B₂O₃; about 15 mol% Na₂O; about 20 mol% MgO; about 10 mol% CaO; and about 5 mol% NaF, KF, CaF₂, SnF₂, or any combination thereof.

15. The glass composition according to claim 1, comprising:
from about 5 mol% to about 10 mol% of CaF₂, SnF₂, NaF, KF, or a combination thereof; and
from about 90 mol% to about 95 mol% of a combination of B₂O₃, Na₂O, MgO, and CaO;
wherein the boron, the magnesium, the combination of sodium and any potassium, and the Ca in the glass composition are present in elemental ratios of about 20: about 4: about 6: about 3, respectively.

16. The glass composition according to claim 15, comprising:
about 50 mol% B₂O₃,
about 15 mol% Na₂O,
about 20 mol% MgO,
about 10 mol% CaO, and
about 5 mol% NaF, KF, CaF₂, SnF₂, or any combination thereof.

17. The glass composition according to claim 15, comprising: about 50 mol% B₂O₃, about 15 mol% Na₂O, about 20 mol% MgO, about 10 mol% CaO, and about 5 mol% CaF₂.

18. The glass composition according to claim 15, comprising: about 48 mol% B₂O₃, about 9 mol% Na₂O, about 19 mol% MgO, about 14 mol% CaO, and about 10 mol% NaF.

19. The glass composition according to any one of claims 1 to 18, wherein the glass composition is a particulate material that comprises particles that are from about 1 to about 50 µm in size.

20. The glass composition according to claim 19, wherein at least 75% of the particles are smaller than 50 µm in size.

21. The glass composition according to claim 19, wherein at least 85% of the particles are smaller than 50 µm in size.

22. The glass composition according to claim 19, wherein at least 95% of the particles are smaller than 50 µm in size.

23. The glass composition according to any one of claims 19 to 22, wherein at least 5% of the particles are smaller than 7 µm in size.

24. The glass composition according to any one of claims 19 to 22, wherein:
at least 5% of the particles are smaller than 35 µm in size,
at least 5% of the particles are smaller than 15 µm in size, and
at least 5% of the particles are smaller than 7 µm in size.

25. The glass composition according to any one of claims 19 to 23, wherein:
at least 5% of the particles are from about 15 µm to about 35 µm in size,
at least 5% of the particles are from about 6 µm to about 15 µm in size, and
at least 5% of the particles are from about 3 µm to about 7 µm in size.

26. The glass composition according to claim 19, wherein:
about 10% of the particles are smaller than 5 µm in size,
about 50% of the particles are smaller than 15 µm in size, and
about 90% of the particles are smaller than 30 µm in size.

27. The glass composition according to any one of claims 19 to 26, wherein the glass composition loses at least 5 mass% within 24 hours when exposed to a buffered saline solution.

28. The glass composition according to any one of claims 19 to 26, wherein the glass composition loses at least 20 mass% within 24 hours when exposed to a buffered saline solution.

29. The glass composition according to any one of claims 19 to 26, wherein the glass composition loses at least 40 mass% within 24 hours when exposed to a buffered saline solution.

30. The glass composition according to any one of claims 19 to 26, wherein the glass composition loses at least 60 mass% within 24 hours when exposed to a buffered saline solution.

31. The glass composition according to any one of claims 19 to 26, wherein the glass composition loses at least 80 mass% within 24 hours when exposed to a buffered saline solution.

32. A toothpaste comprising the glass composition according to any one of claims 19 to 26, such as in an amount of from about 0.5 to about 15 mass% of the toothpaste, preferably in an amount of about 5 mass%.

33. A toothpaste comprising the glass composition according to any one of claims 19 to 26, wherein the toothpaste includes a sufficient amount of the glass composition to result in about 500 ppm (m/m) to about 1,500 ppm (m/m) of the fluoride, such as from about 750 ppm to about 1,500 ppm of the fluoride, or from about 1,000 ppm to about 1,500 ppm of the fluoride.

34. The toothpaste according to claim 32 or 33, wherein the toothpaste further comprises glycerin, such as pure glycerol; sodium lauryl sulphate; silica; Carbopol 940; and a flavoring agent such as spearmint oil.

35. The toothpaste according to claim 34, wherein the toothpaste comprises about 85 mass% glycerol, about 1.2 mass% sodium lauryl sulphate, about 7.5 mass% silica, about 0.5 mass% Carbopol 940, about 1.0 mass% flavoring agent, and about 5.0 mass% of the glass composition.

36. A prophylaxis paste comprising the glass composition according to any one of claims 19 to 26.

37. A prophylaxis paste comprising the glass composition according to any one of claims 19 to 26, wherein the toothpaste includes a sufficient amount of the glass composition to result in about 1,000 ppm (m/m) to about 1,500 ppm (m/m) of the fluoride.

38. A tooth varnish comprising the glass composition according to any one of claims 19 to 26.

39. A tooth varnish comprising the glass composition according to any one of claims 19 to 26, wherein the toothpaste includes a sufficient amount of the glass composition to result in about 1,000 ppm (m/m) to about 5000 ppm (m/m) of the fluoride.

40. Use of the toothpaste according to any one of claims 32 to 35 to at least temporarily reduce pain associated with sensitive teeth.

41. Use of the prophylaxis paste according to claim 36 or 37 to at least temporarily reduce pain associated with sensitive teeth.

42. Use of the tooth varnish according to claim 38 or 39 to at least temporarily reduce pain associated with sensitive teeth.

43. A method of at least temporarily reducing, in an individual, pain associated with sensitive teeth, the method comprising applying:
the toothpaste according to any one of claims 32 to 35,
the prophylaxis paste according to claim 36 or 37, or
the tooth varnish according to claim 38 or 39,
to dentin in the individual.

44. A glass composition according to any one of claims 19 to 26 for desensitizing dentin.

45. The glass composition for desensitizing dentin according to claim 44 for temporarily reducing pain associated with sensitive teeth.

46. A dentin-desensitizing composition comprising:
(i) the glass composition according to any one of claims 19 to 26, 44 and 45; and
(ii) a water-free, orally-compatible carrier.

47. The dentin-desensitizing composition according to claim 46 wherein the orally-compatible carrier is a mouthwash.

48. The dentin-desensitizing composition according to claim 46 wherein the orally-compatible carrier is formulated to mix with a mouthwash.

49. The dentin-desensitizing composition according to claim 46 wherein the orally-compatible carrier is an orally-compatible viscous carrier.

50. The dentin-desensitizing composition according to claim 49 wherein the orally-compatible viscous carrier has a viscosity from about 100 cP at 30° C. to about 150,000 cp at 30° C.

51. The dentin-desensitizing composition according to claim 50 wherein the orally-compatible viscous carrier is a toothpaste, a dental gel, a prophylaxis paste, a tooth varnish, or a bonding agent.

52. The glass composition according to any one of claims 1 to 16, wherein the glass is a bulk glass, for preparing a particulate glass composition according to any one of claims 19 to 26.

53. Use of the toothpaste according to any one of claims 32 to 35 to increase surface enamel microhardness.

54. A method to increase surface enamel microhardness, the method comprising applying the toothpaste according to any one of claims 32 to 35 to enamel in the individual.

55. A glass composition according to any one of claims 19 to 26 for increasing surface enamel microhardness.

56. Use of the toothpaste according to any one of claims 32 to 35 to at least partially remineralize surface enamel.

57. A method to at least partially remineralize surface enamel, the method comprising applying the toothpaste according to any one of claims 32 to 35 to enamel in the individual.

58. A glass composition according to any one of claims 19 to 26 for at least partially remineralizing surface enamel.

59. Use of the toothpaste according to any one of claims 32 to 35 to at least partially occlude one or more dentin tubules.

60. A method to at least partially occlude one or more dentin tubules, the method comprising applying the toothpaste according to any one of claims 32 to 35 to the dentin tubules in an individual.

61. A glass composition according to any one of claims 19 to 26 for at least partially occluding one or more dentin tubules.
